(19) United States
(12) Patent Application Publication
(10) Pub. No.: US 2015/0323520 A1
(43) Pub. Date: Nov. 12, 2015

(54) DIAGNOSIS OF KIDNEY TRANSPLANT ACUTE REJECTION

(71) Applicant: The Trustees of the Stevens Institute of Technology, Hoboken, NJ (US)

(72) Inventors: Jingmao Chi, Jersey City, NJ (US); Henry Du, Short Hills, NJ (US); Peter Tolias, Westfield, NJ (US)

(73) Assignee: The Trustees of the Stevens Institute of Technology, Hoboken, NJ (US)

(21) Appl. No.: 14/705,371

(22) Filed: May 6, 2015

Related U.S. Application Data
(60) Provisional application No. 61/989,956, filed on May 7, 2014.

(51) Int. Cl. G01N 33/493; H01J 49/00; G01J 3/44; G01N 33/50; G01N 21/78

(52) U.S. Cl. CPC G01N 33/493; G01N 33/50; G01N 21/78; G01J 3/44; H01J 49/0036

(57) ABSTRACT

A method for identifying a kidney condition of a patient includes receiving a urine sample from a patient. The patient provides the urine sample a predetermined amount of time after having been administered a dose of a sugar alcohol. The method also includes determining whether the sugar alcohol is present in the urine sample. The method also includes identifying a kidney condition of the patient, if the sugar alcohol is present in the urine sample.

### CROSS-REFERENCE TO RELATED APPLICATION

The present application is a Section 111(a) application relating to and claiming the benefit of commonly owned, co-pending U.S. Provisional Patent Application No. 61/989,956, titled “SERS-ACTIVE FIBER PROBE FOR KIDNEY TRANSPLANT ACUTE REJECTION DIAGNOSIS,” having a filing date of May 7, 2014, which is incorporated by reference herein in its entirety.

### FIELD OF THE DISCLOSURE

The present disclosure relates generally to the field of chemical sensors and related methods of use, and, more particularly, to chemical sensors for use in analysis of biological fluids and medical diagnostics.

### BACKGROUND OF THE DISCLOSURE

Transplanted organs are at risk of acute rejection (“AR”) by the host. Although biopsy of the organ is the present “gold standard” for diagnosing AR, biopsy is expensive, invasive, and typically not performed until weeks or months after transplant. It is known that AR may be accompanied by changes in the concentrations of certain metabolites in the host's body fluids.

### SUMMARY OF THE INVENTION

In an embodiment, the present invention relates to a method for identifying a kidney condition of a patient including receiving a urine sample from a patient. The patient provides the urine sample a predetermined amount of time after having been administered a dose of a sugar alcohol. The method also includes determining whether the sugar alcohol is present in the urine sample and identifying a kidney condition of the patient, if said sugar alcohol is present in said urine sample.

In an embodiment, the determining whether the sugar alcohol is present in the urine sample includes measuring a Raman spectrum for the urine sample using a surface-enhanced Raman scattering sensor and determining that the sugar alcohol is present in the urine sample when the Raman spectrum includes a Raman intensity peak at a Raman shift of about 1360 cm⁻¹.

In an embodiment, the surface-enhanced Raman scattering sensor includes an optic fiber. In an embodiment, the optic fiber includes a tip, and the tip includes plurality of nanoparticles immobilized thereon. In an embodiment, the plurality of nanoparticles is selected from a group consisting of a plurality of silver nanoparticles, a plurality of gold nanoparticles, a plurality of platinum nanoparticles, and a plurality of palladium nanoparticles. In an embodiment, the plurality of nanoparticles is a plurality of silver nanoparticles, and the plurality of silver nanoparticles are immobilized on the tip by coating the tip with a layer of polyallylamine hydrochloride and dipping the coated tip in colloidal silver. In an embodiment, the optic fiber includes one of a silica fiber and a sapphire fiber. In an embodiment, the surface-enhanced Raman scattering sensor includes an optic wafer.

In an embodiment, the determining whether the sugar alcohol is present in the urine sample includes measuring a mass spectrum for the urine sample using a mass spectrometer and determining that the sugar alcohol is present in the urine sample when the mass spectrum includes an intensity peak at a mass-to-charge ratio of about 217. In an embodiment, the mass spectrometer includes an electrospray ionization mass spectrometer.

In an embodiment, the determining whether the sugar alcohol is present in the urine sample comprises testing for the sugar alcohol with a clinical chemistry testing system. In an embodiment, the determining whether the sugar alcohol is present in the urine sample comprises performing a colorimetric analysis for the urine sample. In an embodiment, the determining whether the sugar alcohol is present in the urine sample comprises exposing a reagent test strip to the urine sample.

In an embodiment, the sugar alcohol is selected from a group consisting of mannitol, sorbitol, and xylitol. In an embodiment, the sugar alcohol is mannitol, and the predetermined amount of time is a day after the dose of the sugar alcohol was administered to the patient. In an embodiment, the dose of the sugar alcohol is provided as a part of a kidney transplant surgery, and the kidney condition is acute rejection.

### BRIEF DESCRIPTION OF THE FIGURES

FIG. 1 is an illustration of an exemplary Raman spectroscopy device that may be used to detect acute rejection of a transplanted kidney according to an exemplary embodiment;

FIG. 2 is a chart showing SERS spectra of urine samples of three kidney transplant patients undergoing AR, as measured with a sensor such as the exemplary device of FIG. 1;

FIG. 3 is a chart showing SERS spectra of urine samples of three kidney transplant patients without AR, as measured with a sensor such as the exemplary device of FIG. 1;

FIG. 4 is a chart showing a SERS spectrum of a urine sample of a first kidney patient experiencing graft failure, as measured with a sensor such as the exemplary device of FIG. 1;

FIG. 5 is a chart showing a SERS spectrum of a urine sample of a second kidney patient experiencing graft failure, as measured with a sensor such as the exemplary device of FIG. 1;

FIG. 6 is a chart showing mass spectra of urine samples of three kidney transplant patients undergoing AR, as measured by a mass spectrometer;

FIG. 7 is a chart showing mass spectra of urine samples of three kidney transplant patients without AR, as measured by a mass spectrometer; and

FIG. 8 is a chart showing a mass spectrum of a saline solution of mannitol and a mass spectrum of a urine sample of a kidney transplant patient undergoing AR, as measured by a mass spectrometer.

### DETAILED DESCRIPTION OF HE INVENTION

Transplanted organs are at risk of acute rejection (“AR”) by the host. AR is an immune response of the host to destroy the grafted organ, typically occurs within the first year after transplant, and occurs in about 10% of patients in the United States and at higher rates in the developing world. AR results in both worse clinical outcomes (e.g., in the case of kidney transplants, a return to dialysis, a need for a retransplant, or death) and increased health care costs. Early diagnosis of AR may mitigate both of these results.

A biomarker useful in detecting AR of a transplanted kidney is a patient's level of serum creatinine (sCr). A patient's sCr level is monitored every day during an inpatient hospital stay. Elevated sCr levels indicate a potential AR. However, sCr monitoring is invasive because it is necessary to draw blood from a patient. Additionally, sCr levels sometimes do not rise until one to three months after a patient's development of AR, leading to delayed diagnosis. Further, increases in sCr are not specific to AR, since there are many causes other than AR for an increase in a patient's sCr level. Thus, even when elevated levels of levels of sCr are detected, further diagnosis is required.

Biopsy of the organ is the present “gold standard” for diagnosing AR of a transplanted organ such as a kidney. However, biopsy is expensive, typically costing between $3,000 and $9,000. Additionally, biopsy is an invasive procedure, and therefore carries the risk of complications. Further, biopsy is typically not performed until weeks or months after transplant.

The exemplary embodiments described herein present techniques for diagnosing AR in recipients of kidney transplants that are non-invasive, specific, and effective the day after transplant occurs. The exemplary embodiments will be described herein with reference to the use of specific detection equipment and a specific biomarker. However, it will be apparent to those of skill in the art that the concepts encapsulated by the exemplary embodiments may be adapted for application through the use of differing equipment and analytes from those specifically described herein. Broadly, the exemplary embodiments may be performed by receiving a urine sample from a patient who has been the recipient of a kidney transplant and evaluating the sample for the presence of an analyte that is indicative of AR.

In a first embodiment, a urine sample may be evaluated through the use of surface-enhanced Raman scattering (“SERS”) spectroscopy. SERS is an ultra-sensitive molecular detection technique that is often effective at below the parts per million (“ppm”) level, or even at the single-molecule level, and is often used for high-concentration or bulk analysis. Raman scattering is scattering of photons that occurs when light interacts with a vibrating molecule. SERS spectroscopy employs a sensor to observe a spectrum

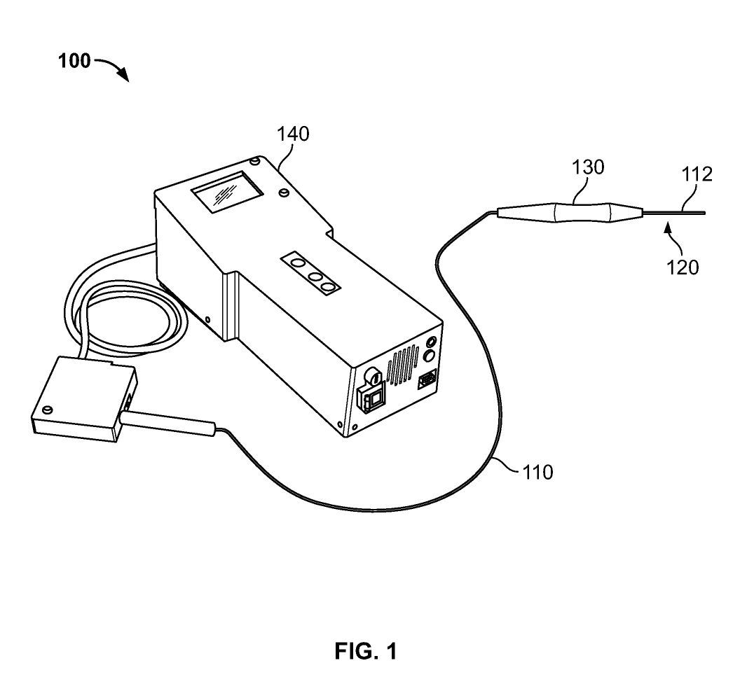

FIG. 1 illustrates an exemplary device 100 for observing SERS spectra of test samples. The device 100 includes an optical fiber 110 having a tip 112. Optical fiber 110 is functionalized with silver nanoparticles 120 at tip 112. In the embodiment shown in FIG. 1, the optical fiber 110 is fitted with a handle 130 and is coupled to a compact, hand-held sensor probe 140 to form device 100. In other embodiments, Raman spectroscopy may be performed by coupling optical fiber 110 to spectroscopy devices other than the hand-held device shown in FIG. 1.

In an exemplary method of making the device 100, a cleaved distal end of optical fiber 110 is functionalized by coating tip 112 with a layer of polyallylamine hydrochloride (“PAH”), by, for example, dipping the tip of the fiber into a solution of PAH. The PAH-coated tip 112 is then dipped into colloidal silver, thus immobilizing the silver nanoparticles 120 on the fiber tip 112. The optical fiber 110 may be of any type (e.g., a conventional sapphire or silica optical fiber). Other means of immobilizing silver nanoparticles 120 on an optical fiber 110 may be employed by those having ordinary skill in the art. In an embodiment, other elemental nanoparticles (e.g., gold, platinum, palladium) may be used in place of silver. In an embodiment, nanoparticles (e.g., silver nanoparticles) may be immobilized on a planar substrate (e.g., a silica wafer) rather than an optic fiber, and SERS spectra may be determined through the use of the substrate with nanoparticles immobilized thereon.

The device 100 is described with specific reference to techniques for diagnosing AR of a transplanted kidney by using the sensor to detect indicators of AR in urine samples of kidney transplant patients who may be undergoing AR. In addition to the diagnosis of kidney transplant AR, urinary biomarkers that indicate other diseases may also be detected by the device 100 through the same or a similar process.

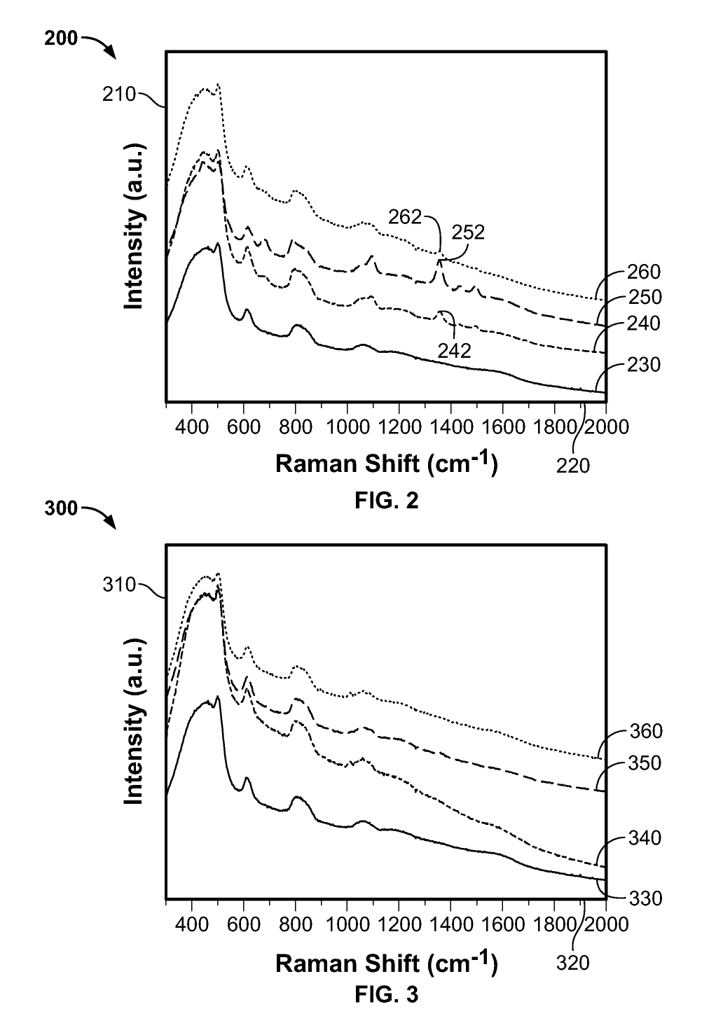

FIG. 2 is a graph 200 showing SERS spectra for patients who have received kidney transplants and are experiencing AR. The graph 200 plots Raman intensity, in arbitrary units (“a.u.”), along a vertical axis 210 against Raman shift, in cm⁻¹, along a horizontal axis 220. As a baseline, the chart 200 includes a background spectrum 230 that may be measured when a SERS sensor (e.g., the device 100 of FIG. 1) is immersed in water. The chart 200 also includes three SERS spectra 240, 250, 260 that may be measured when the same SERS sensor is immersed in urine samples of three AR patients. It may be observed that each of the three SERS spectra 240, 250, 260 includes a corresponding peak 242, 252, 262, at a value of about 1360 cm⁻¹ along the horizontal axis 220.

FIG. 3 is a graph 300 showing SERS spectra for patients who have received kidney transplants and are not experiencing AR. The graph 300 plots Raman intensity, in arbitrary units (“a.u.”), along a vertical axis 310 against Raman shift, in cm⁻¹, along a horizontal axis 320. As a baseline, the chart 300 includes a background spectrum 330 that may be measured when a SERS sensor (e.g., the device 100 of FIG. 1) is immersed in water (e.g., the same background spectrum as is shown in FIG. 2). The chart 300 also includes three SERS spectra 340, 350, 360 that may be measured when the same SERS sensor is immersed in urine samples of three kidney transplant recipients who are not experiencing AR. It may be observed that the three SERS spectra 340, 350, 360 lack a peak at a value of 1360 cm⁻¹ along the horizontal axis 320.

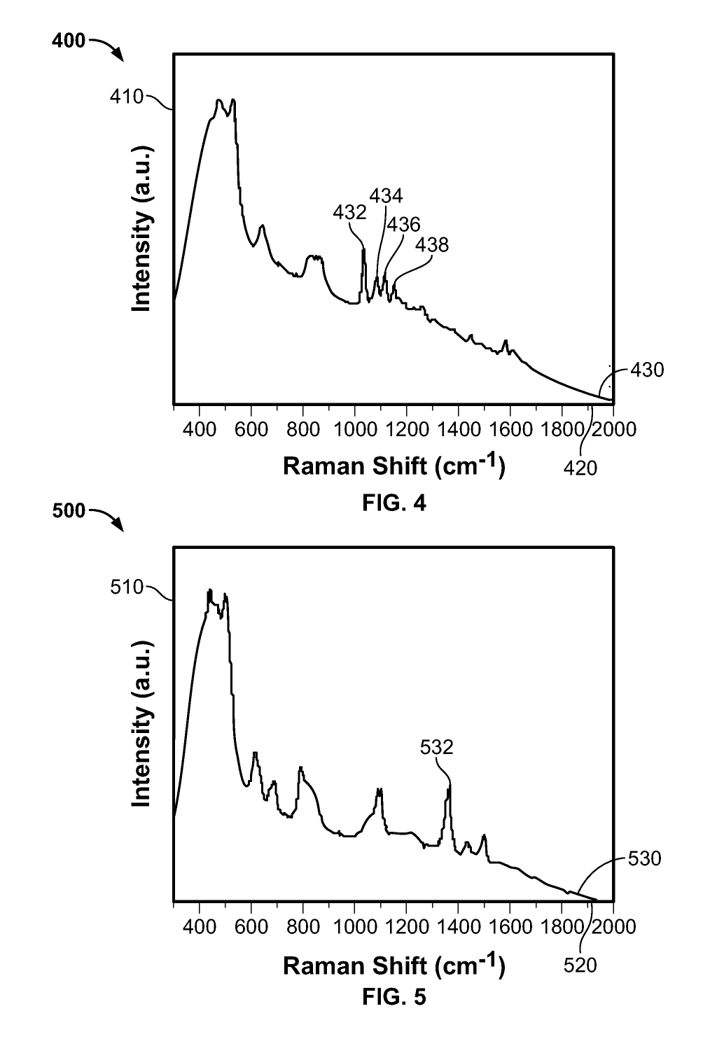

FIG. 4 is a graph 400 showing an SERS spectrum for a patient who has received a kidney transplant and is experiencing graft failure. The graph 400 plots Raman intensity, in arbitrary units (“a.u.”), along a vertical axis 410 against Raman shift, in cm⁻¹, along a horizontal axis 420. The chart 400 includes a SERS spectrum 430 measured when a SERS sensor (e.g., the device 100 of FIG. 1) is immersed in a urine sample of a kidney transplant recipient who is experiencing graft failure. It may be observed that the SERS spectrum includes peaks 432, 434, 436, 438, at corresponding values 1003 cm⁻¹, 1088 cm⁻¹, 1121 cm⁻¹, and 1152 cm⁻¹ along the horizontal axis 420, all of which are absent in the SERS spectra 340, 350, 360 of patients whose transplants were successful.

FIG. 5 is a graph 500 showing an SERS spectrum for a patient who has received a kidney transplant and is experiencing graft failure. The graph 500 plots Raman intensity, in arbitrary units (“a.u.”), along a vertical axis 510 against Raman shift, in cm⁻¹, along a horizontal axis 520. The chart 500 includes a SERS spectrum 530 measured when a SERS sensor (e.g., the device 100 of FIG. 1) is immersed in a urine sample of a kidney transplant recipient who is experiencing graft failure. It may be observed that the SERS spectrum 530 includes a peak 532 at a value of 1360 cm⁻¹ along the horizontal axis 520, similar to the peaks 242, 252, 262 of the corresponding spectra 240, 250, 260 of patients experiencing AR shown in FIG. 2.

Based on the above, in an embodiment, evaluation may be performed through SERS spectroscopy of a urine sample provided by a patient the day after receiving a kidney transplant. If the patient's SERS spectrum includes a peak at 1360 cm⁻¹, it may be inferred that the patient is experiencing AR. Depending on clinical preference, this may be taken as a conclusive diagnosis, or may be used as a basis for scheduling a biopsy to arrive at a conclusive diagnosis. In either case, a determination may be made as soon as the day after the transplant, rather than after the delay inherent in other diagnostic techniques. Conversely, if the patient's SERS spectrum lacks a peak at 1360 cm⁻¹, it may be inferred that the patient is not experiencing AR.

The exemplary diagnostic techniques described above may also be performed through the use of different diagnostic equipment from the SERS device described above with reference to FIG. 1. In an embodiment, a sample may be evaluated through the use of mass spectrometry. Mass spectrometry is widely used in identification, verification and quantitation of biomarkers because mass spectrometers measure mass to a high degree of accuracy and can provide the chemical structure of analytes. Electrospray ionization mass spectrometry (“ESI-MS”) is one exemplary type of mass spectrometry that may be used for this purpose, and the exemplary embodiments will be described hereinafter with specific reference to ESI-MS. However, those of skill in the art will understand that other types of mass spectrometry equipment may be used in the performance of the exemplary techniques without departing from the broader principles encapsulated by the exemplary embodiments. Other types of mass spectrometry that may be used for this purpose include matrix-assisted laser desorption/ionization mass spectrometry, fast atom bombardment mass spectrometry, chemical ionization mass spectrometry, atmospheric-pressure chemical ionization mass spectrometry, gas chromatography mass spectrometry, liquid chromatography mass spectrometry, capillary electrophoresis mass spectrometry, ion mobility spectrometry mass spectrometry, and other types of mass spectrometry not specifically mentioned herein.

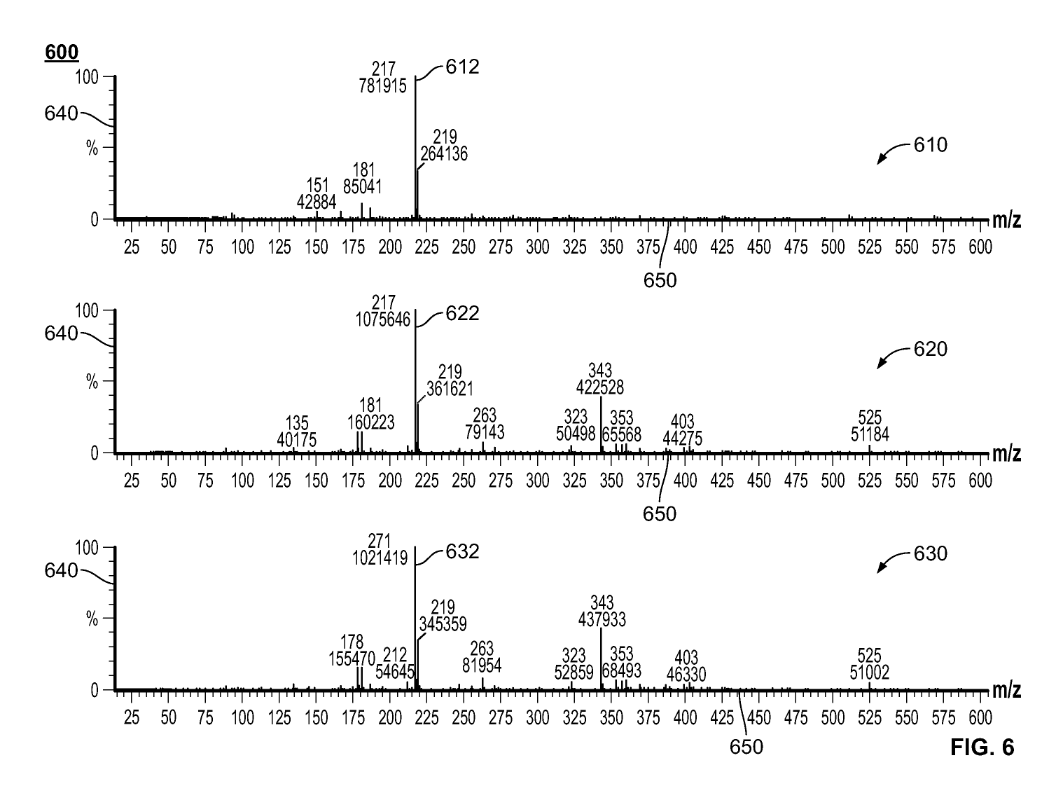

FIG. 6 is a graph 600 showing three ESI-MS spectra 610, 620, 630 for patients who have received kidney transplants and are experiencing AR. The spectra 610, 620, 630 plot intensity, an expression of relative abundance expressed as a percentage, along a vertical axis 640 against mass-to-charge ratio m/z, an expression of the ratio of atomic mass to charge number, which is a dimensionless quantity, along a horizontal axis 650. It may be observed that each of the ESI-MS spectra 610, 620, 630 shown in graph 600 includes an intense peak 612, 622, 632 at an m/z value of 217.

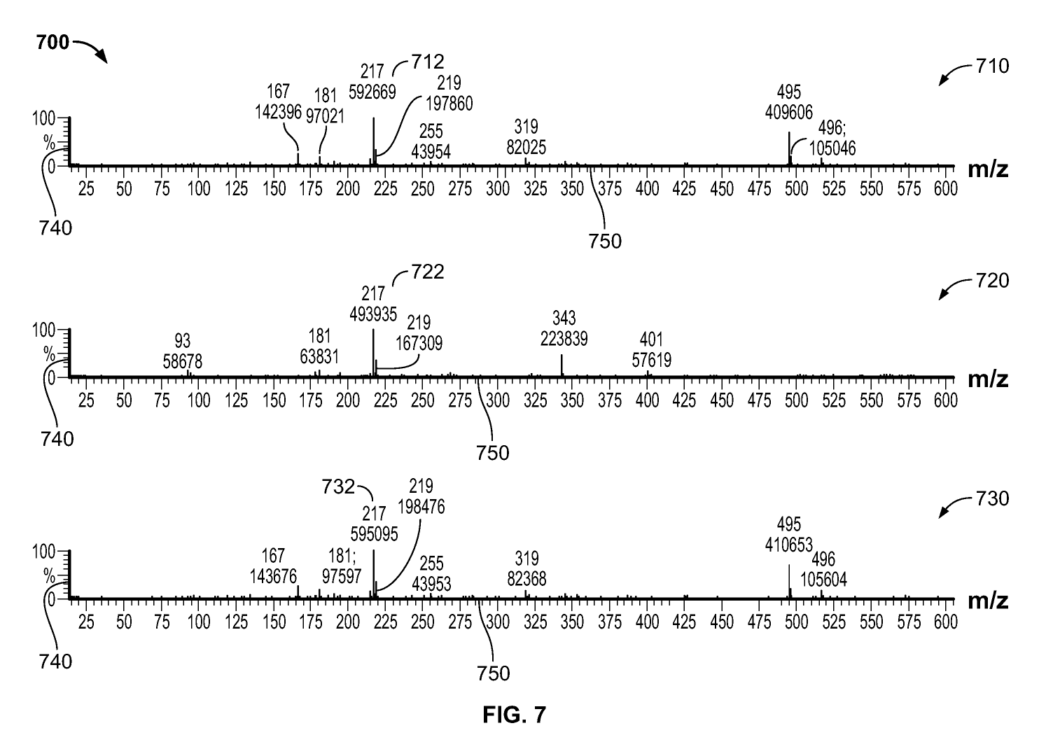

FIG. 7 is a graph 700 showing three ESI-MS spectra 710, 720, 730 for patients who have received kidney transplants and are not experiencing AR. The spectra 710, 720, 730 plot intensity, in percentage, along a vertical axis 770 against mass-to-charge ratio m/z along a horizontal axis 780. It may be observed that each of the ESI-MS spectra 710, 720, 730, shown in graph 700 includes a much smaller peak 712, 722, 732, at an m/z value of 217. More specifically, it may be observed that the peaks 612, 622, 632 shown in FIG. 6 and observed for patients who are experiencing AR may be on the order of two times to ten times the intensity of the peaks 712, 722, 732 observed for patients who are not experiencing AR.

Based on the above, in an embodiment, evaluation may be performed through mass spectrometry of a urine sample provided by a patient the day after receiving a kidney transplant. If the patient's ESI-MS spectrum includes an intense peak at an m/z value of 217, it may be inferred that the patient is experiencing AR. Depending on clinical preference, this may be taken as a conclusive diagnosis, or may be used as a basis for scheduling a biopsy to arrive at a conclusive diagnosis. In either case, a determination may be made as soon as the day after the transplant, rather than after the delay inherent in other diagnostic techniques. Conversely, if the patient's ESI-MS spectrum includes a small peak at an m/z value of 217, it may be inferred that the patient is not experiencing AR.

Referring back to FIG. 6 and considering the graph 600 in greater detail, it may be inferred that the mass indicated by the peaks 612, 622, 632 at the mass-to-charge ratio of 217 are characteristic of mannitol. This identification may be made through the use of a lock mass in ESI-MS. A lock mass is a suitable known compound introduced to an ion source to provide real-time recalibration by correcting m/z shifts arising from instrument drift. In the present case, leu-enkephalin was used as the lock mass. Several known compounds with m/z values close to 217 were tested and an accuracy of within 5 millidaltons was achieved. Based on this level of accuracy, a more precise m/z value of 217.0480±0.0002 may be determined. The standard m/z value of mannitol chloride adduct negative ion is 217.0479. Thus, the peaks 612, 622, 632 shown at m/z value 217 in ESI-MS spectra 610, 620, 630 of AR patients in FIG. 6 may be identified as mannitol with a negatively charged chloride ion.

A large dose of mannitol (e.g., 20 grams) is routinely given to patients during kidney transplant surgery to dilate blood vessels and tubules within the transplanted organ to promote blood flow to the transplanted organ and thereby prime it to function. The administered dose of mannitol may also promote urination by the transplant recipient. All patients whose SERS or mass spectra are shown in FIGS. 2-7 were administered mannitol during kidney transplant surgery. Transplant patients whose surgery is successful metabolize the mannitol quickly, and the mannitol is barely detectable in such patients' urine 24 hours after surgery. Patients who are experiencing AR do not metabolize mannitol well, resulting in high levels of mannitol in the urine of such patients 24 hours after surgery. Therefore, it may be apparent to those of skill in the art that mannitol in a patient's urine 24 hours after kidney transplant surgery is a prognostic indicator of AR.

Similarly, patients who are experiencing graft failure do not metabolize mannitol well. For example, the graft failure SERS spectrum 530 shown in FIG. 5 includes the same peak 532 as is shown for AR SERS spectra 242, 252, 262. The graft failure SERS spectrum 430 shown in FIG. 4 does not include an analogous peak at 1360 cm⁻¹, but includes four peaks 432, 434, 436, 438 not shown in the SERS spectra 340, 350, 360 for healthy transplants. Thus, it may be inferred that a measured SERS spectrum containing such peaks (e.g., peaks similar to peaks 432, 434, 436, 438) may be indicative of graft failure; such peaks may indicate metabolism byproducts of mannitol or biomarkers other than mannitol. It may also be inferred that a measured SERS spectrum containing a peak at 1360 cm⁻¹ may also be indicative of graft failure.

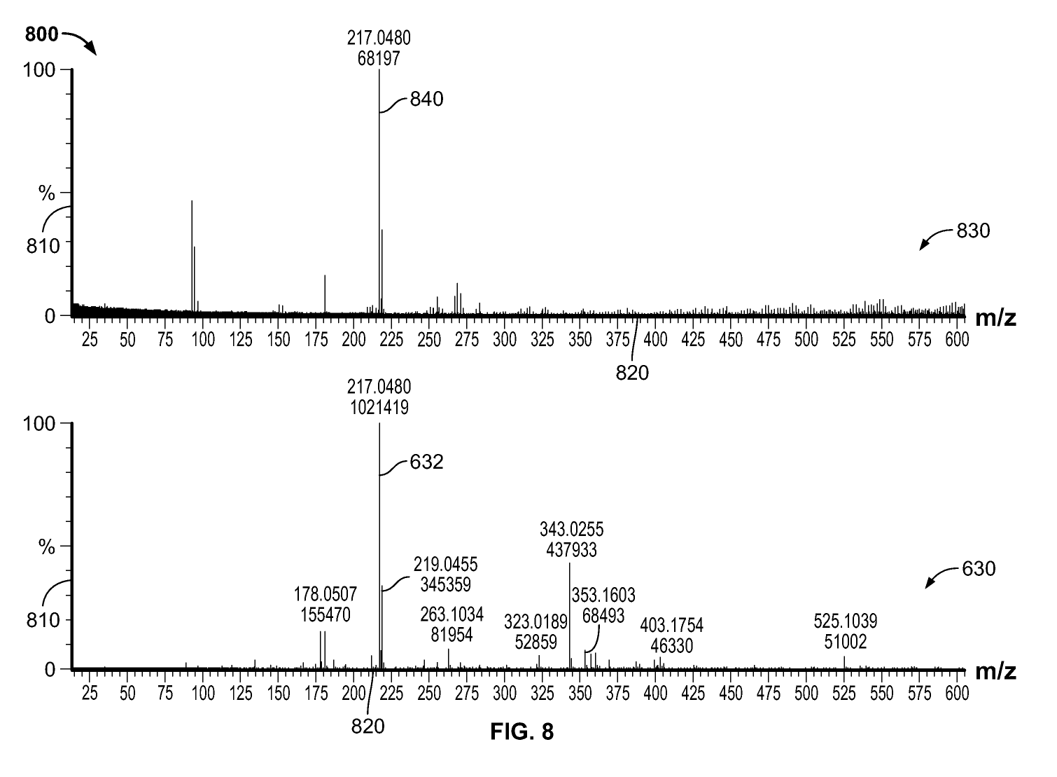

Referring now to FIG. 8, to test the inference that the peaks noted above (e.g., peaks 242, 252, 262 of FIG. 2 and peaks 612, 622, 632 of FIG. 6) indicate the presence of mannitol in the test samples for which said peaks are observed, an ESI-MS spectrum for a saline solution of mannitol is shown. Graph 800, like graphs 600 and 700, plots intensity, in percentage, along a vertical axis 810 against mass-to-charge ratio m/z along a horizontal axis 820. Graph 800 includes an ESI-MS spectrum 830 for a saline solution of mannitol. For comparison purposes, graph 800 also includes ESI-MS spectrum 630 for a patient who has received a kidney transplant and is experiencing AR, which was previously shown in FIG. 6. It may be observed that the ESI-MS spectrum 830 includes an intense peak 840 at an m/z value of 217, collocated with the peak 632 of the ESI-MS spectrum 630. Therefore, it may be concluded that the peaks noted above (e.g., peaks 242, 252, 262 of FIG. 2 and peaks 612, 622, 632 of FIG. 6) indicate the presence of mannitol in the test samples for which said peaks are observed. Consequently, the presence of mannitol in said test samples may indicate that a transplanted kidney that is manifesting AR may fail to properly metabolize mannitol that was administered during transplant surgery; conversely, the absence of mannitol in a test sample may indicate that a transplanted kidney has properly metabolized mannitol and that no mannitol remains in the patient's urine sample.

It should be noted that the above describes characteristics of urine samples provided by kidney transplant recipients the day after transplant surgery. Therefore, it will be apparent to those of skill in the art that urine samples received sooner (e.g., the day of surgery) may not provide useful results because even patients whose transplanted kidneys are not experiencing AR will not yet have had sufficient time to metabolize mannitol administered during transplant surgery. Consequently, evaluation of urine samples received from such patients may reveal the presence of mannitol, which may be falsely indicative of AR. Conversely, urine samples received significantly later (e.g., a week after surgery) also may not provide useful results because even patients whose transplanted kidneys are experiencing AR will have been able to metabolize mannitol administered during transplant surgery. Thus, evaluation of urine samples received from such patients may reveal the absence of mannitol, which may be falsely indicative of a lack of AR. Therefore, a urine sample to be used for evaluation according to the exemplary embodiments should be provided a predetermined time after the administration of mannitol to the patient (e.g., a day later) in order to ensure that accurate results are provided.

As described above, detection of AR through the use of the exemplary embodiments (e.g., by testing a patient's urine sample with a surface-enhanced Raman spectroscopy device or a mass spectrometer) may provide advantages over existing techniques. Testing of a urine sample is non-invasive and involves no risk of complications to the patient. The diagnosis made may be specific to the patient's kidney function, as opposed to detection based on elevated serum creatinine levels that may be due to another cause. Testing according to the exemplary embodiments is inexpensive. Detection may occur the day after transplant surgery, providing ample time for clinicians to plan a further course of treatment, thereby reducing overall costs and improving clinical outcomes.

The present invention has been described above with reference to specific exemplary embodiments. However, those of skill in the art will understand that the broader principles of the exemplary embodiments may be applied in various manners not described in detail above. In one exemplary variation, different diagnostic equipment may be used to detect an analyte, such as mannitol, in a urine sample provided by a transplant patient. In one embodiment, a clinical chemistry testing system used to test samples for the presence of various other components (e.g., glucose, cholesterol, sodium, etc.) may be configured and implemented to perform an assay to test for the presence of mannitol. In another embodiment, a colorimetric analysis may be performed to test for the presence of mannitol. In another embodiment, a reagent strip may be exposed to a patient's urine sample to test for the presence of mannitol.

In another exemplary variation, evaluation of a urine sample for the presence of mannitol may be performed to evaluate the health of a non-transplanted kidney. For example, a dose of mannitol (e.g., 20 grams, as is commonly used during kidney transplant surgery) may be administered to a patient whose kidney health is to be verified. A predetermined amount of time later (e.g., 24 hours later), the patient may be asked to provide a urine sample. The patient's urine sample may be evaluated using the same exemplary techniques described above with reference to a urine sample provided by a recipient of a kidney transplant, and criteria used to diagnose AR in a kidney transplant recipient (e.g., a peak at a point on a Raman spectrum or a mass spectrum as described above) may be used to diagnose poor health of a non-transplanted kidney.

In another exemplary variation, an analyte other than mannitol may be considered. For example, a different sugar alcohol, such as sorbitol or xylitol, may also be administered to a patient for similar purposes to those described above with reference to mannitol, and a patient who has received a different sugar alcohol may provide a urine sample to be evaluated in substantially the same manner described above. It will be apparent to those of skill in the art that the appropriate predetermined time after administration at which the patient should provide a urine sample may vary for different analytes and that the appropriate predetermined time may be determined by one of skill in the art without undue experimentation.

It will be understood that the embodiments of the present invention described herein are merely exemplary and that a person skilled in the art may make many variations and modifications without departing from the spirit and scope of the invention. All such variations and modifications are intended to be included within the scope of the invention.

What is claimed is:

1. A method for identifying a kidney condition of a patient, comprising:
receiving a urine sample from a patient, the patient providing said urine sample a predetermined amount of time after having been administered a dose of a sugar alcohol;
determining whether said sugar alcohol is present in said urine sample; and
identifying a kidney condition of the patient, if said sugar alcohol is present in said urine sample.

2. The method of claim 1, wherein said determining whether said sugar alcohol is present in said urine sample comprises:
measuring a Raman spectrum for said urine sample using a surface-enhanced Raman scattering sensor; and
determining that said sugar alcohol is present in said urine sample when said Raman spectrum includes a Raman intensity peak at a Raman shift of about 1360 cm⁻¹.

3. The method of claim 2, wherein said surface-enhanced Raman scattering sensor comprises an optic fiber.

4. The method of claim 3, wherein said optic fiber includes a tip, and wherein said tip includes plurality of nanoparticles immobilized thereon.

5. The method of claim 4, wherein said plurality of nanoparticles is selected from a group consisting of a plurality of silver nanoparticles, a plurality of gold nanoparticles, a plurality of platinum nanoparticles, and a plurality of palladium nanoparticles.

6. The method of claim 4, wherein said plurality of nanoparticles is a plurality of silver nanoparticles, and wherein said plurality of silver nanoparticles are immobilized on said tip by coating said tip with a layer of polyallylamine hydrochloride and dipping said coated tip in colloidal silver.

7. The method of claim 3, wherein said optic fiber comprises one of a silica fiber and a sapphire fiber.

8. The method of claim 2, wherein said surface-enhanced Raman scattering sensor comprises a planar substrate.

9. The method of claim 1, wherein said determining whether said sugar alcohol is present in said urine sample comprises:
measuring a mass spectrum for said urine sample using a mass spectrometer; and
determining that said sugar alcohol is present in said urine sample when said mass spectrum includes an intensity peak at a mass-to-charge ratio of about 217.

10. The method of claim 9, wherein said mass spectrometer comprises one of an electrospray ionization mass spectrometer, a matrix-assisted laser desorption/ionization mass spectrometer, a fast atom bombardment mass spectrometer, a chemical ionization mass spectrometer, an atmospheric-pressure chemical ionization mass spectrometer, a gas chromatography mass spectrometer, a liquid chromatography mass spectrometer, a capillary electrophoresis mass spectrometer, and an ion mobility spectrometry mass spectrometer.

11. The method of claim 1, wherein said determining whether said sugar alcohol is present in said urine sample comprises testing for said sugar alcohol with a clinical chemistry testing system.

12. The method of claim 1, wherein said determining whether said sugar alcohol is present in said urine sample comprises performing a colorimetric analysis for said urine sample.

13. The method of claim 1, wherein said sugar alcohol is selected from a group consisting of mannitol, sorbitol, and xylitol.

14. The method of claim 1, wherein said sugar alcohol is mannitol, and wherein said predetermined amount of time is a day after said dose of said sugar alcohol was administered to the patient.

15. The method of claim 1, wherein said dose of said sugar alcohol is provided during a kidney transplant surgery, and wherein said kidney condition is acute rejection.
